(19) United States
(12) Patent Application Publication
(10) Pub. No.: US 2008/0268038 A1
(43) Pub. Date: Oct. 30, 2008

(54) Compositions and Approaches for Increasing Diet Induced Thermogenesis, Inducing Weight Loss and Maintaining Muscle Mass and Strength

(76) Inventor: Robert R. Wolfe, Little Rock, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/110,016

(22) Filed: Apr. 25, 2008

Related U.S. Application Data
(60) Provisional application No. 60/914,073, filed on Apr. 26, 2007.

(51) Int. Cl. A61K 9/48; A61K 31/4172; A61K 31/195; A61K 31/522; A61K 36/752; A61P 3/04; A61K 31/60; A61K 31/4188; A61K 31/5375

(52) U.S. Cl. 424/452; 514/385; 514/561; 514/263.31; 514/162; 514/393; 514/231.2; 424/736

(57) ABSTRACT

Provided are compositions and methods for increasing diet induced thermogenesis. Typically, the compositions are comprised of L-histidine, L-isoleucine, L-leucine, L-lysine, L-methionine, L-phenylalanine, L-valine and L-threonine.

### CROSS REFERENCE TO RELATED APPLICATIONS

This application claims benefit of U.S. Provisional Patent Application No. 60/914,073, filed Apr. 26, 2007, the entire disclosure of which is incorporated herein by reference.

### GOVERNMENTAL RIGHTS IN INVENTION

This work was supported in part by the United States Public Health Service, National Institutes of Health, National Center for Research Resources Grant M01-RR-00073. The U.S. Government, therefore, may have certain rights in this invention.

### BACKGROUND OF THE INVENTION

1. Field of the Invention

The present invention generally relates to compositions and methods for increasing diet induced thermogenesis. In particular, a composition comprising eight essential amino acids.

2. Description of Related Art

Obesity has reached epidemic proportions globally, and is a major contributor to the global burden of chronic disease and disability. While the causes of obesity are complex, major contributors include the increased consumption of high-calorie, nutrient-poor foods and the lack of physical activity. When food energy intake exceeds energy expenditure, the excess energy is stored as fat. Measures to prevent or combat obesity generally include promoting the eating of healthy food and/or increasing physical activity. However, such measures have limited compliance.

Another approach to the prevention of obesity is the development of supplements or foodstuffs that would increase energy expenditure. Daily energy expenditure comprises three components: basal metabolic rate, diet induced thermogenesis (DIT), and activity induced thermogenesis (AIT). DIT generally refers to the stimulation of energy expenditure following nutrient ingestion. It is well known among those knowledgeable of the components of daily energy expenditure that each nutrient affects DIT differently. For example, protein increases DIT about 20-30%, carbohydrate increases DIT about 5-10%, and fat increases DIT only 0-3%. Although DIT represents a relatively small component of total energy expenditure, it may be of potential significance in the maintenance of an individual's energy balance over a prolonged period of time. Thus, a supplement or a foodstuff that increases DIT could play a critical role in a weight maintenance or weight reduction program. There is a need for a supplement or a foodstuff that provides the greatest DIT per Kcal ingested while also providing important nutritive value.

Maintenance of muscle mass during weight loss is important in several respects. Maintenance of muscle mass and strength preserves physical function, which is particularly important in obese elderly individuals who have a decreased muscle mass before weight loss and cannot afford to loose any more muscle without adversely affecting mobility to a significant extent. Secondly, muscle mass is continually renewed by a simultaneous synthesis of new protein and breakdown of old protein. This process is associated with energy expenditure. Maintained or increased muscle mass, as a result of stimulation of muscle protein synthesis, will contribute to the resting energy expenditure due to the energy cost of accelerated synthesis and breakdown of muscle protein. Consequently, it would be useful to develop a dietary supplement that not only increased DIT but also stimulated muscle protein synthesis.

Resistance exercise can also help to preserve muscle mass and strength during weight loss. Incorporation of resistance exercise into a weight loss program using a dietary supplement that stimulates DIT as well as muscle protein synthesis may therefore result in beneficial results in terms of both weight loss as well as maintenance of muscle mass and muscle function. Finally, the degree of DIT induced by a nutrient that stimulates muscle protein synthesis will be dependent on the pattern of ingestion. It therefore may be useful to utilize a pattern of ingestion that maximizes the DIT and the muscle protein synthesis of the nutrient.

### SUMMARY OF THE INVENTION

The following is a summary of the invention in order to provide a basic understanding of some of the aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems know to those of skill in the art, described herein, among other things, is a composition of matter comprising the essential amino acids (EAAs) L-histidine, L-isoleucine, L-leucine, L-lysine, L-methionine, L-phenylalanine, L-valine and L-threonine.

In an embodiment of such composition the concentration of L-leucine is not less than about 18% of the EAAs and not more than about 40% by weight of the EAAs composition.

In another embodiment of the composition, the concentration of L-histidine ranges from about 8% to about 14% of EAAs by weight of the composition, the concentration of L-isoleucine ranges from about 8% to about 14% of EAAs by weight of the composition, the concentration of L-lysine ranges from about 12% to about 18% of EAAs by weight of the composition, the concentration of L-methionine ranges from about 3% to about 5% of EAAs by weight of the composition, the concentration of L-phenylalanine ranges from about 10% to about 18% of EAAs by weight of the composition, the concentration of L-valine may be between 8-14% of EAAs by weight of the composition, and the concentration of L-threonine ranges from about 12% to about 18% of EAAs by weight of the composition.

In another embodiment of the composition, the concentration of L-histidine is about 12.4% of EAAs by weight of the composition, the concentration of L-isoleucine is about 11.8% of EAAs by weight of the composition, the concentration of L-leucine is about 21.1% of EAAs by weight of the composition, the concentration of L-lysine is about 17.4% of EAAs by weight of the composition, the concentration of L-methionine is about 3.5% of EAAs by weight of the composition, the concentration of L-phenylalanine is about 17.4% of EAAs by weight of the composition, and the concentration of L-threonine is about 16.5% of EAAs by weight of the composition.

A still further embodiment of the composition additionally comprises L-valine at a concentration that ranges from about 9% to about 12% of EAAs by weight of the composition.

A still further embodiment of the composition additionally comprises an agent selected from the group consisting of: synephrine, Citrus aurantium extract, phenylpropanolamine, caffeine, aspirin, and a combination thereof.

A still further embodiment of the composition additionally comprises an agent selected from the group consisting of: sibutramine, phentermine, diethylpropion, mazindol, and phendimetrazine.

In a still further embodiment of the composition, the amino acids of the composition are in free form and are incorporated into a food product selected from a group consisting of: a drink, a frozen treat bar, a snack bar, and a bakery product.

In a still further embodiment of the composition, the amino acids of the composition are in salt form and are incorporated into a food product selected from a group consisting of: a drink, a frozen treat bar, a snack bar, and a bakery product.

There is also described herein, a capsule comprising: a core material comprising L-histidine, L-isoleucine, L-leucine, L-lysine, L-methionine, L-phenylalanine, L-valine and L-threonine; and a shell wall that encapsulates the core material.

In an embodiment of the capsule, the concentration of L-histidine in the core material ranges from about 9% to about 13% (w/w), the concentration of L-isoleucine in the core material ranges from about 9% to about 12% (w/w), the concentration of L-leucine in the core material ranges from about 18% to about 40% (w/w), the concentration of L-lysine in the core material ranges from about 13% to about 18% (w/w), the concentration of L-methionine in the core material ranges from about 3% to about 4% (w/w), the concentration of L-phenylalanine in the core material ranges from about 13% to about 18% (w/w), the concentration of L-valine in the core material ranges from about 10% to about 12% (w/w), and the concentration of L-threonine in the core material ranges from about 13% to about 17% (w/w).

In a further embodiment of the capsule, the concentration of L-histidine in the core material is about 12.4% (w/w), the concentration of L-isoleucine in the core material is about 11.8% (w/w), the concentration of L-leucine in the core material is about 21.1% (w/w), the concentration of L-lysine in the core material is about 17.4% (w/w), the concentration of L-methionine in the core material is about 3.5% (w/w), the concentration of L-phenylalanine in the core material is about 17.4% (w/w), and the concentration of L-threonine in the core material is about 16.5% (w/w).

In a still further embodiment of the capsule, the amino acids of the core material are in free form.

In a still further embodiment of the capsule, the amino acids of the core material are in salt form.

In a further embodiment of the capsule, the core material further comprises at least one excipient.

In a still further embodiment of the capsule, the core material further comprises an agent selected from the group consisting of: synephrine, Citrus aurantium extract, phenylpropanolamine, caffeine, aspirin, and a combination thereof.

In a still further embodiment of the capsule, the core material further comprises an agent selected from the group consisting of: sibutramine, phentermine, diethylpropion, mazindol, and phendimetrazine.

In a still further embodiment of the capsule, the shell wall is a material selected from the group comprising soft gelatin, hard gelatin, and a polymer.

There is also described herein, a method of use of the composition described above in a program to loose weight and/or to transform body composition to favor increased muscle mass and decreased fat mass.

There is also described herein, a method to combine ingestion of the composition with a resistance exercise routine to result in weight loss and/or transformation of body composition to favor increased muscle mass and strength and decreased fat mass.

There is also described herein, a method to provide the above composition in 4-5 doses per day in amounts not to exceed 15 g of EAAs per dose to maximize the thermogenic response and the stimulation of muscle protein synthesis over the entire day.

There is also described herein, a method to provide the composition described above immediately before resistance exercise to maximize the anabolic response to the exercise and to the EAAs.

There is also described herein, a purified polypeptide, the polypeptide comprising L-histidine residues, L-isoleucine residues, L-leucine residues, L-lysine residues, L-methionine residues, L-phenylalanine residues, L-valine residues, and L-threonine residues.

In an embodiment of the purified polypeptide, the percentage of L-histidine residues in the polypeptide ranges from about 9% to about 13%, the percentage of L-isoleucine residues in the polypeptide ranges from about 9% to about 12%, the percentage of L-leucine residues in the polypeptide ranges from about 18% to about 40%, the percentage of L-lysine residues in the polypeptide ranges from about 13% to about 18%, the percentage of L-methionine residues in the polypeptide ranges from about 3% to about 4%, the percentage of L-phenylalanine residues in the polypeptide ranges from about 13% to about 18%, the percentage of L-valine residues in the polypeptide ranges from about 10% to about 12%, and the percentage of L-threonine residues in the polypeptide ranges from about 13% to about 17%.

In a further embodiment of the purified polypeptide, the polypeptide is incorporated into a food product.

In a still further embodiment of the purified polypeptide, the food product is selected from the group consisting of: a drink, a frozen treat bar, a snack bar, and a bakery product.

There is still further described herein, a method for increasing diet induced thermogenesis in a subject, the method comprising administering to the subject a composition comprised of L-histidine, L-isoleucine, L-leucine, L-lysine, L-methionine, L-phenylalanine, L-valine and L-threonine.

In an embodiment of the method for increasing diet induced thermogenesis in a subject, the concentration of L-histidine in the composition ranges from about 9% to about 13% (w/w), the concentration of L-isoleucine in the composition ranges from about 9% to about 12% (w/w), the concentration of L-leucine in the composition ranges from about 18% to about 40% (w/w), the concentration of L-lysine in the composition ranges from about 13% to about 18% (w/w), the concentration of L-methionine in the composition ranges from about 3% to about 4% (w/w), the concentration of L-phenylalanine in the composition ranges from about 13% to about 18% (w/w), the concentration of L-valine in the composition ranges from about 10% to about 12% (w/w), and the concentration of L-threonine in the composition ranges from about 13% to about 17% (w/w).

In a further embodiment of method for increasing diet induced thermogenesis in a subject, an amount of the composition ranging from about 7 g to about 15 g is administered to the subject.

In a still further embodiment of method for increasing diet induced thermogenesis in a subject, the composition is administered from about 1 hour to about 3 hours after a meal.

In a still further embodiment of method for increasing diet induced thermogenesis in a subject, the composition is administered about 2 hours after a meal.

In a still further embodiment of method for increasing diet induced thermogenesis in a subject, administration of the composition is selected from the group consisting of: one, two, and three times per day.

There is still further described herein a method for increasing diet induced thermogenesis in a subject wherein the subject is less than about 60 years of age, and the concentration of L-histidine in the composition is about 12.8% (w/w), the concentration of L-isoleucine in the composition is about 12.2% (w/w), the concentration of L-leucine in the composition is about 18.5% (w/w), the concentration of L-lysine in the composition is about 18.0% (w/w), the concentration of L-methionine in the composition is about 3.6% (w/w), the concentration of L-phenylalanine in the composition is about 17.9% (w/w), and the concentration of L-threonine in the composition is about 17.0% (w/w).

In an embodiment of the method for increasing diet induced thermogenesis in a subject, the subject is at least 60 years of age, and the concentration of L-histidine in the composition is about 9.4% (w/w), the concentration of L-isoleucine in the composition is about 9.0% (w/w), the concentration of L-leucine in the composition is about 40.0% (w/w), the concentration of L-lysine in the composition is about 13.2% (w/w), the concentration of L-methionine in the composition is about 2.7% (w/w), the concentration of L-phenylalanine in the composition is about 13.2% (w/w), and the concentration of L-threonine in the composition is about 12.5% (w/w).

In a further embodiment of the method for increasing diet induced thermogenesis in a subject, the increased thermogenesis is due to increased muscle protein synthesis.

In a still further embodiment of the method for increasing diet induced thermogenesis in a subject, the increased thermogenesis is due to increased ATP production.

There is still further described herein a method for promoting weight loss in a subject, the method comprising administering to the subject a composition comprised of L-histidine, L-isoleucine, L-leucine, L-lysine, L-methionine, L-phenylalanine, L-valine and L-threonine.

In an embodiment of the method of promoting weight loss in a subject, the concentration of L-histidine in the composition ranges from about 9% to about 13% (w/w), the concentration of L-isoleucine in the composition ranges from about 9% to about 12% (w/w), the concentration of L-leucine in the composition ranges from about 18% to about 40% (w/w), the concentration of L-lysine in the composition ranges from about 13% to about 18% (w/w), the concentration of L-methionine in the composition ranges from about 3% to about 4% (w/w), the concentration of L-phenylalanine in the composition ranges from about 13% to about 18% (w/w), the concentration of L-valine in the composition ranges from about 10% to about 12% (w/w), and the concentration of L-threonine in the composition t ranges from about 13% to about 17% (w/w).

In a further embodiment of the method of promoting weight loss in a subject, the concentration of L-histidine in the composition is about 12.4% (w/w), the concentration of L-isoleucine in the composition is about 11.8% (w/w), the concentration of L-leucine in the composition is about 21.1% (w/w), the concentration of L-lysine in the composition is about 17.4% (w/w), the concentration of L-methionine in the composition is about 3.5% (w/w), the concentration of L-phenylalanine in the composition is about 17.4% (w/w), and the concentration of L-threonine in the composition is about 16.5% (w/w).

In a still further embodiment of the method of promoting weight loss in a subject, an amount of the composition ranging from about 7 g to about 15 g is administered to the subject.

In a still further embodiment of the method of promoting weight loss in a subject, the composition is administered from about 1 hour to about 3 hours after a meal.

In a still further embodiment of the method of promoting weight loss in a subject, the composition is administered about 2 hours after a meal.

In a still further embodiment of the method of promoting weight loss in a subject, administration of the composition is selected from the group consisting of: one, two, and three times per day.

In a still further embodiment of the method of promoting weight loss in a subject, the composition further comprises an agent selected from the group consisting of: synephrine, Citrus aurantium extract, phenylpropanolamine, caffeine, aspirin, and a combination thereof.

In a still further embodiment of the method of promoting weight loss in a subject, the composition further comprises an agent selected from the group consisting of: sibutramine, phentermine, diethylpropion, mazindol, and phendimetrazine.

In a still further embodiment of the method of promoting weight loss in a subject, the method further comprises increasing physical activity of the subject.

In a still further embodiment of the method of promoting weight loss in a subject, weight loss in the subject is promoted by increased DIT.

In a still further embodiment of the method of promoting weight loss in a subject, increased DIT leads to decreased body fat and increased lean body mass.

These and other embodiments of the invention are described in more detail herein.

### BRIEF DESCRIPTION OF THE DRAWINGS

FIG. 1 is a bar graph depicting changes in the thermogenic response. Plotted are the changes in energy expenditure (i.e., DIT) as compared to baseline over four hours (area under curve, AUC) following ingestion of 15 g of a mix of essential amino acids (EAAs) or a placebo. Values are means ±SEM.

FIG. 2 is a bar graph illustrating the changes in energy expenditure over time. The percent change in energy expenditure in response to 15 gm of EAAs relative to placebo is plotted for intervals of time during the study.

FIG. 3 shows the individual and interactive effects of resistance exercise and EAAs. Bars represent the balance between muscle protein synthesis and breakdown, i.e., the net loss (below zero) or gain (above zero) on muscle protein.

FIG. 4 is a bar graph representing the effect of ingestion of various doses of EAAs on the rate of muscle protein synthesis in normal human volunteers.

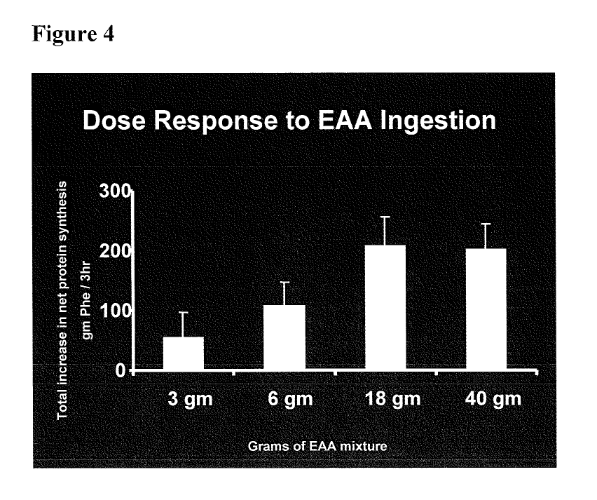

FIG. 5 represents the difference in muscle protein synthesis when EAAs are ingested before or at various times after performance of resistance exercise.

### DETAILED DESCRIPTION OF PREFERRED EMBODIMENTS

The following detailed description illustrates by way of example and not by way of limitation. Before describing the preferred embodiments, it is important to explain several key terms as used herein.

The term, “diet induced thermogenesis” (DIT) as used herein, generally refers to the stimulation of energy expenditure following nutrient ingestion. DIT is one component of daily energy expenditure, the other two components being basal metabolic rate and activity induced thermogenesis.

The term, “essential amino acids” (EAA), as used herein, generally refers to those amino acids that cannot be synthesized de novo by an organism at a rate sufficient to meet requirement, and therefore, must be provided in the diet. Nine amino acids are generally regarded as essential for humans. They are: L-histidine, L-isoleucine, L-leucine, L-lysine, L-methionine, L-phenylalanine, L-threonine, L-tryptophan, and L-valine.

The term, “excipient,” as used herein, refers to an inactive substance that may be combined with the amino acid compositions described herein to make various dosage forms.

Described herein, among other things, are compositions that increase DIT. These compositions are comprised of a blend of EAAs in which the proportion of each amino acid is tailored to optimize muscle protein synthesis, and consequently, increase DIT while maintaining or increasing muscle mass. It has been discovered that these EAAs increase DIT by more than 40%, which is the highest value for any nutrient (see the examples). Also provided are methods for increasing DIT and promoting weight loss/weight management by administering an amino acid composition of the type described. There is also discussed incorporation of a plan for ingestion in multiple doses of specified amounts of the discussed compositions to maximize the effect on thermogenesis and muscle protein synthesis. There is also discussed the use of the composition in conjunction with exercise to maximize gain in muscle mass and strength.

In one embodiment, there is provided a composition comprising eight EAAs. An EAA is generally described as one that cannot be synthesized de novo by the organism to which the composition is provided, and therefore, must be provided in the diet. The compositions generally comprise the minimal number of EAAs, whose proportions are optimized for maximal stimulation of muscle protein synthesis and DIT.

The compositions discussed herein generally comprise of the following eight EAAs: L-histidine, L-isoleucine, L-leucine, L-lysine, L-methionine, L-phenylalanine, L-valine and L-threonine. It is widely recognized among those skilled or knowledgeable in dietary processes that L-leucine plays an especially important role in the stimulation of muscle protein synthesis. Furthermore, it has also been shown that a higher level of L-leucine is required in elderly individuals for the optimal stimulation of muscle protein synthesis (Katsanos et al. 2006, Am J Physiol Endocrinol Metab 291:E381-87). Accordingly, the concentration of L-leucine in the compositions can and will vary depending upon a variety of factors, including the age and health status of an individual. In alternative embodiments, the compositions could include only 7 of the 8 EAAs above not including L-leucine. Alternatively, the composition could also include L-Tryptophan to have 9 EAAs.

In one embodiment, the concentration of L-leucine in a composition may range from 18% to about 40% of the total EAAs by weight. In another embodiment, the concentration of L-leucine may range from 18% to about 20% of the EAAs by weight. In still another embodiment, the concentration of L-leucine may range from 21% to about 25% of the EAAs by weight. In yet another embodiment, the concentration of L-leucine may range from 26% to about 30% of EAAs by weight. In another alternate embodiment, the concentration of L-leucine may range from 31% to about 35% of EAAs by weight. In a further embodiment, the concentration of L-leucine may range from 36% to about 40% of EAAs by weight.

The relative ratios of the other seven amino acids are adjusted accordingly such that DIT is optimally stimulated. The concentration of L-histidine in the composition may be between 8%, and 14% of EAAs by weight. The concentration of L-isoleucine in the composition may be between 8%, and 14% of EAAs by weight. The concentration of L-lysine in the composition may be between 12 and 18% of EAAs by weight. The composition of L-valine may be between 8-14% of EAAs by weight. The concentration of L-methionine in the composition may be between 3-5%% of EAAs by weight. The concentration of L-phenylalanine in the composition may be between about 10-18% of EAAs by weight. The concentration of L-threonine in the composition may be between about 12-18% of EAAs by weight.

In a preferred embodiment, the EAA composition comprises about 12.4% by weight of L-histidine, about 11.8% by weight of L-isoleucine, about 21.1% by weight of L-leucine, about 17.4% by weight of L-lysine, about 3.5% by weight of L-methionine, about 17.4% by weight of L-phenylalanine, about 14.9% L-valine and about 16.5% by weight of L-threonine.

In another preferred embodiment, a composition for a healthy individual under the age of about 60 years, the EAA composition comprises about 12.8% by weight of L-histidine, about 12.2% by weight of L-isoleucine, about 18.5% by weight of L-leucine, about 18.0% by weight of L-lysine, about 3.6% by weight of L-methionine, about 17.9% by weight of L-phenylalanine, and about 17.0% by weight of L-threonine.

In yet another preferred embodiment, a composition for an individual over the age of about 60 years, the EAA composition comprises about 8.2% by weight of L-histidine, about 7.8% by weight of L-isoleucine, about 40.0% by weight of L-leucine, about 11.0% by weight of L-lysine, about 2.7% by weight of L-methionine, about 10.2% by weight of L-phenylalanine, about 11.3% by weight of L-threonine, and about 8.8% by weight of L-valine.

The amino acids of the composition may be produced by a fermentation method, in which microorganisms synthesize an amino acid that is then isolated. Alternatively, the amino acids may be produced by a hydrolysis method, in which proteins are degraded and the constituent amino acids are then isolated. The amino acids may also be enzymatically or chemically synthesized. Further, the amino acids may be modified or derivatized, for example with acetyl or hydroxyl groups, to improve solubility or other properties. By no means are any of these processes required. It is contemplated that the amino acids may be produced by any method known and recognized by those skilled in the art. In general, the amino acids will be of food grade quality or pharmaceutical grade quality. Each of the amino acids of the composition may be in a free form, a salt form, complexed with a metal ion, combination thereof or any other form recognized by those skilled in the art.

The composition may be a blend of individual amino acids, in the relative proportions detailed above. Alternatively, the composition blend could be in the form of a mixture of proteins, and/or peptides, and/or amino acids. Alternatively, the amino acids of the composition may be linked together by peptide bonds to form a polypeptide. Typically, polypeptides are more stable than free amino acids, and a polypeptide form may mask the unpalatability of some of the free amino acids. A polypeptide is comprised of the same relative proportion of the eight amino acids. Accordingly, a purified polypeptide comprises about 9% to about 13% of L-histidine residues, about 9% to about 12% of L-isoleucine residues, L about 18% to about 40% of L-leucine residues, about 13% to about 18% of L-lysine residues, about 3% to about 4% of L-methionine residues, about 13% to about 18% of L-phenylalanine residues, about 13% to about 17% of L-threonine residues, and about 7-11% L-valine residues.

The length of the purified polypeptide may vary. In general, shorter polypeptides are easier and less costly to manufacture. Thus, the shorter purified polypeptide generally will be preferred. The preferred shorter purified polypeptide may have about 32 residues comprising about four L-histidine residues, about three L-isoleucine residues, about six L-leucine residues, about five L-lysine residues, about one L-methionine residue, about five L-phenylalanine residues, about five L-threonine residues, and about three L-valine residues. The purified polypeptides may be synthesized using standard protein synthesis procedures, which are well known in the art.

The compositions may also optionally comprise an appetite suppressant. The appetite suppressant may be an over-the-counter drug. Non-limiting examples of non-prescription appetite suppressants include, without limitation, phenylpropanolamine, citrus aurantium extract, synephrine, tyramine, octopamine, and Hoodia. Non-prescription appetite suppressants may be used in combination with a methylxanthine, such as caffeine and/or, a salicylate, such as aspirin. The appetite suppressant may also be a prescription drug. Non-limiting suitable examples of prescription appetite suppressants include, without limitation, sibutramine, phentermine, diethylpropion, mazindol, and phendimetrazine.

Supplemental minerals may also be included in the amino acid compositions. Suitable minerals may include one or more minerals or mineral sources. Non-limiting examples of minerals include, without limitation, chloride, sodium, calcium, iron, chromium, copper, iodine, zinc, magnesium, manganese, molybdenum, phosphorus, potassium, and selenium. Suitable forms of any of the foregoing minerals include, but are not limited to, soluble mineral salts, slightly soluble mineral salts, insoluble mineral salts, chelated minerals, mineral complexes, non-reactive minerals such as carbonyl minerals, reduced minerals, and combinations thereof.

The amino acid compositions may also optionally comprise vitamins. The vitamins may be fat-soluble or water soluble vitamins. Suitable vitamins may include, but are not limited to, vitamin C, vitamin A, vitamin E, vitamin B12, vitamin K, riboflavin, niacin, vitamin D, vitamin B6, folic acid, pyridoxine, thiamine, pantothenic acid, and biotin. The forms of the vitamin may include, but are not limited to, salts of the vitamin, derivatives of the vitamin, compounds having the same or similar activity of a vitamin, and metabolites of a vitamin.

The compositions may also comprise at least one excipient, as such term has been previously defined. Non-limiting examples of suitable excipients include a buffering agent, a preservative, a stabilizer, a binder, a compaction agent, a lubricant, a dispersion enhancer, a disintegration agent, a flavoring agent, a sweetener, a coloring agent, and combinations of any of these agents.

In one embodiment, the excipient is a buffering agent. Non-limiting examples of suitable buffering agents include sodium citrate, magnesium carbonate, magnesium bicarbonate, calcium carbonate, and calcium bicarbonate.

The excipient may comprise a preservative. Suitable examples of preservatives include, but are not limited to, antioxidants, such as alpha-tocopherol or ascorbate, and antimicrobials, such as parabens, chlorobutanol, phenol, or combinations thereof.

In another embodiment, the excipient may be a binder. Suitable binders include, but are not limited to, starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a lubricant. Suitable non-limiting examples of lubricants include, but are not limited to, magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, light mineral oil.

The excipient may be a dispersion enhancer. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In yet another embodiment, the excipient may be a disintegrant. The disintegrant may be a non-effervescent disintegrant. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. The disintegrant may be an effervescent disintegrant. Suitable effervescent disintegrants include sodium bicarbonate in combination with citric acid, and sodium bicarbonate in combination with tartaric acid.

The excipient may include a flavoring agent. Flavoring agents will generally be incorporated into an outer layer and may be chosen from synthetic flavor oils and flavoring aromatics and/or natural oils, extracts from plants, leaves, flowers, fruits, combinations thereof, or any other flavor recognized by those knowledgeable in the art. By way of non-limiting example, these may include cinnamon oils, oil of wintergreen, peppermint oils, clover oil, hay oil, anise oil, eucalyptus, vanilla, citrus oil, such as lemon oil, orange oil, grape and grapefruit oil, and fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Depending upon the embodiment, it may be desirable to provide a coloring agent in the outer layer in addition to or instead of flavoring. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the compositions described herein depending on the embodiment.

The weight fraction of the excipient or combination of excipients in the formulation may be about 30% or less, about 25% or less, about 20% or less, about 15% or less, about 10% or less, about 5% or less, about 2%, or about 1% or less of the total weight of the amino acid composition.

The compositions may be formulated into a variety of forms and administered by a number of different means. The compositions may be administered orally, rectally, or parenterally, in formulations containing conventionally acceptable carriers, adjuvants, among other vehicles for administration recognized by those skilled in the art. The term, parenteral, as used herein, includes subcutaneous, intravenous, intramuscular, or intrasternal injection, or infusion techniques. In an exemplary embodiment, the compositions described herein are administered orally.

Solid dosage forms for oral administration may include, but are not limited to, capsules, tablets, caplets, pills, troches, lozenges, powders, and granules. A capsule typically comprises a core material comprising a composition and a shell wall that encapsulates the core material. The core material may be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Some of the above mentioned polymers may also function as taste-masking agents

Tablets, pills, and the like may be compressed, multiply compressed, multiply layered, and/or coated. The coating may be single or multiple. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

Alternatively, powders or granules may be incorporated into a food product. The food product may be a drink. Non-limiting examples of a suitable drink includes fruit juice, a fruit drink, an artificially flavored drink, an artificially sweetened drink, a carbonated beverage, a sports drink, a liquid diary product, a shake, and so forth. The food product may also be a solid foodstuff. Suitable examples of a solid foodstuff include a food bar, a snack bar, a cookie, a brownie, a muffin, a cracker, an ice cream bar, a frozen yogurt bar, and the like.

The compositions may also be in liquid dosage forms for oral administration. Liquid dosage forms include, but are not limited to, aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents.

The compositions may be utilized to increase DIT in a subject. As shown in the following examples, the amino acid compositions increase DIT because of the increased energy cost of the resultant stimulation of muscle protein synthesis and associated processes. Thus, the compositions may be utilized to promote weight loss and/or weight management in a subject.

The method comprises administering to the subject a composition comprising L-histidine, L-isoleucine, L-leucine, L-lysine, L-methionine, L-phenylalanine, L-valine and L-threonine, in proportions such as those detailed above previously. The amount of the composition administered to the subject can and will vary depending upon several factors, including the age and health of the subject. The amount administered may be about 7, 8, 9, 10, 11, 12, 13, 14, or 15 g. In one embodiment, about 7 g of a low leucine composition may be administered to a subject. In another embodiment, about 15 g of a low leucine composition may be administered to a subject. The subject in these two embodiments may be less than about 60, 50, 40, 30, or 20 years of age. In an alternate embodiment, about 7 g of a high leucine composition may be administered to a subject older than about 60 years of age.

The frequency of administration can and will vary depending on the application. In one embodiment, the amino acid composition may be administered one time per day. In an alternate embodiment, the amino acid composition may be administered two times per day. In yet another embodiment, the amino acid composition may be administered three times per day. In still another embodiment, the amino acid composition may be administered four or more times per day.

The timing of administration may also vary. As detailed in the examples, the peak response of energy expenditure generally occurs during the first hour after administration, and the response remains elevated for at least four hours. Accordingly, the amino acid composition may be administered from about one hour to about three hours after a meal. In a preferred embodiment, the amino acid composition may be administered about two hours after a meal.

Since the amino acid compositions discussed previously are designed to be optimized for humans to which they are administered, the subject will generally be a human. The subject may be young, middle-aged, or elderly. The subject may be healthy or have a condition, such as cardiovascular disease, hypertension, osteoporosis, diabetes, metabolic disorder, cancer, and the like. The subject may be of normal weight, slightly overweight, or extremely overweight.

In some embodiments of the method, the amino acid compositions may be administered to a subject to promote weight loss or improve body composition. For example, the subject may lose weight by decreasing body fat and increasing lean body mass. Alternatively, the subject may remain at about the same weight, while decreasing the proportion of body fat and increasing the proportion of lean body mass. In some embodiments, the amino acid composition may further comprise an appetite suppressant, as discussed above. Additionally, the method may further comprise increasing the physical activity of the subject to facilitate the process of losing weight in addition to administration of the compounds.

The amino acid composition will be given up to 4 times per day in doses sufficient to maximally stimulate muscle protein synthesis, as shown in the example. Additionally, the composition may be given shortly before the individual performs resistance exercise. The resistance exercise may comprise a series of multiple repetitions at 80% of the maximal amount of weight that can be lifted for that particular exercise.

The EAAs composition will generally be provided as part of an overall program to loose weight and improve body composition, favoring decreased fat mass and increased muscle mass and strength. The overall program will include optimal dosing of the EAAs, as well as combination with an exercise program. The exercise program may include either aerobic and/or resistance exercise, and in either case ingestion of the EAA prior to the exercise will be part of the program.

### EXAMPLES

The following examples illustrate various embodiments of the composition and methods discussed herein.

### Example 1

### Amino Acid Induced Thermogenesis in Young Healthy Humans

The following study was designed to determine the DIT of 15 g of a mixture of EAAs previously demonstrated to maximally stimulate muscle protein synthesis. It was hypothesized that DIT would be increased as result of the energy cost associated with the stimulation of muscle protein synthesis by the EAAs.

### (a) Methods

Subjects. In accordance with the policies of the Institutional Review Board for the Protection of Human Subjects and the General Clinical Research Center (GCRC) approval committee of the University of Texas Medical Branch, informed written consent was obtained from 10 young healthy adults ages 21-37 years of age (mean age 29±7). The gender and physical characteristics are depicted in Table 1. The subjects (5 males and 5 females) were non-smokers, had no history of metabolic disease, and refrained from physical exercise and alcohol consumption 72 hours prior to the measurement of resting energy expenditure (REE). Female participants had a pregnancy test to rule out pregnancy

TABLE 1
Demographic and Physical Characteristics of Subjects.

| | Means | SD |
|---|---|---|
| Gender (F/M) | 5/5 | |
| Age (yrs) | 29 | 7 |
| Height (cm) | 168 | 5 |
| Weight (Kg) | 74 | 11 |
| Lean Body Mass (Kg) | 52 | 8.2 |
| Fat body Mass (Kg) | 22 | 7.0 |

Experimental Design. Subjects arrived the day of the study after a 12 hour fast. Vital signs, height and weight while wearing a gown were obtained before starting the study. REE was determined in the basal state and in response to the ingestion of 15 gm of 1) an EAA mixture (EAAs; provided by AminoScience Laboratories, Ajinomoto Co. Kawasaki, JP) and 2) a placebo (PL). The order in which the two drinks were given was randomized. There was a washout period of at least 7 days between the two parts of the study. The treatment drink comprises a mixture of EAA (see Table 2) dissolved in 240 ml of an artificially-sweetened drink (Crystal Light™), and the placebo drink was an equal amount of the artificially sweetened drink alone (caloric equivalent=5 Kcals). It was hypothesized that the 15 g EAAs would stimulate energy expenditure as compared to the placebo. The investigators were blinded to the assignment until both parts of the study were completed.

TABLE 2
Composition of the EAA Supplement.

| Amino acid | Amount (g) | % Total |
|---|---|---|
| Histidine | 1.64 | 10.88 |
| Isoleucine | 1.56 | 10.34 |
| Leucine | 2.79 | 18.50 |
| Lysine | 2.30 | 15.25 |
| Methionine | 0.46 | 3.05 |
| Phenylalanine | 2.30 | 15.25 |
| Threonine | 2.20 | 14.59 |
| Tryptophan | 0.10 | 0.66 |
| Valine | 1.73 | 11.47 |
| Total | 15.08 g | 100.00 |

Measurement of Energy Expenditure. Upon arrival in the GCRC, each participant was asked to lie flat on a bed in a dark room for a minimum of 45 minutes to minimize any effect of prior activity or anxiety. The baseline (basal) REE was then determined over two 10-minute periods, separated by 30 minutes by indirect calorimetry using a Sensormedics V2990 metabolic system. Following completion of the basal measurements, the GCRC nursing staff administered either the treatment or placebo drinks. Following consumption of the drink, energy expenditure was measured by indirect calorimetry using a Sensormedics V2990 metabolic system for ten minute sampling intervals beginning at 20, 60, 90, 120, 150, 180, 210 and 240 minutes. Means were taken for the intervals following the 20 and 60, 90 and 120, 150 and 180, and the 210 and 240 values. A convenience break of 20 minutes was permitted to minimize stiffness, fidgeting, boredom and allow the use of the restroom following the 120 minute measurement segment. The first two minutes of each collection period was excluded from data analysis to ensure that measurements were obtained in the steady state. Upon completion of the study, body composition was determined by DEXA analysis.

Data Analysis Oxygen consumption (VO2) and carbon dioxide production (VCO2) were measured by indirect calorimetry. Energy expenditure, carbohydrate and fat oxidation were calculated as described by Frayan (citation). Nutrient induced thermogenesis was calculated as described by Vernet et al. (Am J Physiol Endocrinol Metab 1987, 253:E65-E71) in order to account for the percentage increase in thermogenesis relative to the metabolizable energy ingested. Values of energy expenditure were calculated at each time point, and the area under the curve for the 240 minute experimental period was also calculated. The area under the curve was expressed in relation to the basal value for each individual. DIT was calculated relative to the baseline value and relative to the value of the placebo trial. Data are expressed in means and standard error of the means.

The calculation of the estimated energy expenditure due to stimulation of muscle protein synthesis following EAA ingestion was based on the average leg body mass (LBM) of 8.5 kg (Table 1). It was assumed that the muscle in one leg represented approximately 20% of the total muscle mass and that 26% of the leg LBM was protein-bound amino acids. To calculate the total rate of muscle protein synthesis, previous work had revealed the fractional synthetic rate of muscle protein increased by an average of 0.04%/h following the ingestion of 15 grams of EAAs. To calculate the energy cost of muscle protein synthesis, it was assumed that (1) on average 1 mole of amino acids (AA)=150 g of AA; (2) the cost of synthesizing 1 mol of AA equals 4 ATP; and (3) the hydrolysis of 1 mol of ATP releases 20 Kcal of energy. The formulae are expressed as:

Total bound AA content of muscle (gm)=Leg LBM (grams)×5×0.26 (1)

Incorporation of AAs into muscle protein (gm/h)=(1)×FSR (%/h) (2)

Energy cost of stimulated muscle protein synthesis (Kcal/h)=(2)×1 mol/150 g×4 m ATP/1 mol×20 Kcal (3)

Total energy expenditure due to stimulated muscle protein synthesis (Kcal)=(3)×4 h (4)

There are other processes associated with protein synthesis that require energy, such as RNA synthesis and amino acid transport, and these were also considered in the calculations. Estimates of the energetic costs of these processes vary, and range up to 50% of the cost of peptide bond formation. Thus, the minimal and maximal estimates of energy cost were estimated, with the minimal cost excluding any contribution other than peptide bond formation, and the maximal cost including the maximal estimated cost of RNA synthesis and amino acid transport associated with the stimulation in synthesis (i.e., 50%).

Statistics. The principal outcome variable was the total response of energy expenditure (area under the curve for 4 hours) in the EAA group vs the placebo. Based on the expectation of an increase in energy expenditure in the EAA group due to stimulation of muscle protein synthesis, a one-tailed test paired t-test was used, with p<0.05 being considered statistically significant.

### (b) Results

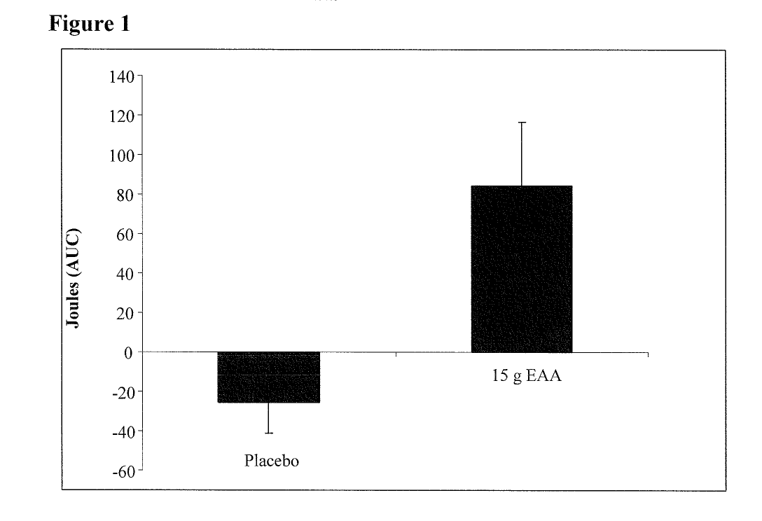

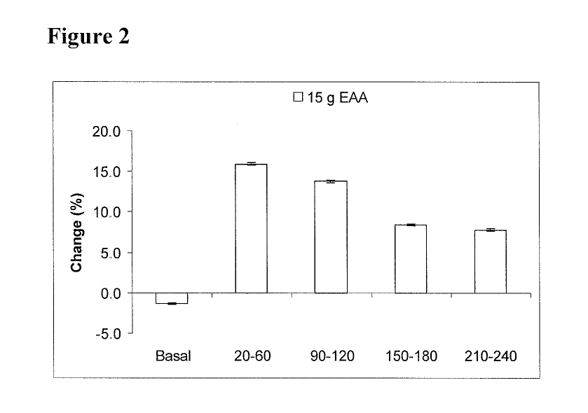

The baseline REE was 52.6±2.16 Kcal/h in the placebo trial and 51.8±1.92 Kcal/h in the EAA trial. Ingestion of EAAs caused increases in both VO2 and VCO2, which translated to a significant increase in the energy expenditure. The overall DIT (area under curve above the baseline value of REE for 4 hours) for EAA was 20.25±7.72 Kcals, as compared to −6.1±3.71 Kcals for the placebo (FIG. 1). The peak response of energy expenditure was in the first hour after ingestion of EAAs, although the value was still elevated over the placebo value after 4 hours (FIG. 2). REE was increased 15.9±0.15% above the baseline value over the first hour, and REE was increased 13.8±0.13% above the baseline value over the second hour. The DIT was 43±13% of the energy ingested in the EAA trial. No significant differences were identified in either fat or carbohydrate oxidation

The calculated estimate of the energy cost of muscle protein synthesis alone was 9.4 Kcal, and the value rose to 14.1 Kcal when the associated contribution of the RNA synthesis and amino acid transport was considered. These values were approximately 40-70% of the thermogenic response to the EAAs, depending on whether the response to the placebo was subtracted from the response to EAAs (yielding a net increase in REE of 26.3 Kcal) or if only the response above baseline to the EAA was considered (20.2 Kcal) and if the maximal or minimal cost of protein synthesis was considered.

### (c) Discussion

The principal finding of this study is that ingestion of a relatively small amount of EAAs significantly stimulated energy expenditure in healthy young volunteers. Energy expenditure was increased by over 20% during the first hour as compared to the response to the placebo. The overall DIT (area under the curve above the baseline value of resting energy expenditure for 4 hours) was equal to 43% of ingested energy. This is a greater DIT than previously reported for any nutrient. This response can be attributed in large part to the unique ability of EAAs to stimulate muscle protein synthesis.

Although DIT represents a relatively small component (approximately 15%) of total energy expenditure, it may nonetheless be of potential significance in the maintenance of energy balance over a prolonged time. For example, if the total energy expenditure of an individual is 2500 Kcal per day, then DIT would be expected to be approximately 375 Kcal per day. A 3% drop (to 12%) or increase (to 18%) in the relative contribution of DIT to total energy expenditure would therefore mean a gain or loss of approximately 75 Kcal per day, assuming constant caloric intake. Extrapolated over a year, this would translate to a gain or loss of almost 8 pounds of fat, since 1 pound of fat stores 3500 Kcal of energy. In the case of the response to EAAs documented in the current investigation, REE was increased by 12.5% over the 4 hours of measurement. Repeated 3 times per day, this would mean a total increase of energy expenditure of 6% per day, or approximately 75 Kcal/day in the subjects. The potential benefit of EAA ingestion as part of a weight-maintenance or weight-reduction program is thus obvious, particularly since muscle protein synthesis is maximally stimulated by the EAAs.

The underlying assumption of the calculation cited above for the long-term effect of repeated EAA ingestion on energy balance is that other energy intake would need to be reduced an amount equal to the caloric value of the EAAs (60 Kcals per dose). Further, the DIT response to other nutrients deleted from the diet would need to be less than the DIT of the EAAs. The latter point could be easily accomplished, as the DIT of the EAAs is greater than anything previously investigated. The DIT of protein, expressed as a percent of energy intake, has rarely been reported to be in excess of about 20%, and the DIT of carbohydrate is generally below about 5%. In contrast, the DIT of EAAs was over 40% in the current study. If the EAAs were substituted for dietary fat, the DIT of the EAAS would directly translate to net negative energy balance, since fat has virtually no DIT.

These findings suggest that regular ingestion of supplemental EAAs (e.g., 3×15 gm/day=180 Kcals) into a weight maintenance/loss program should effectively stimulate muscle protein synthesis while maximizing DIT, and consequently, total energy expenditure. Increased muscle protein synthesis and increased energy expenditure should result in improved body composition and more effective weight management.

### Example 2

### Interaction of Exercise and EAAs

This study was designed to study the interactive effects of exercise and amino acids on the rates of muscle protein synthesis and the net balance between these processes. The later parameter, the net protein balance, reflects the gain or loss of muscle protein.

### (a) Methods

Twelve normal untrained men were studied at rest and then in response to either amino acids alone, exercise alone, or exercise plus amino acids. Leg muscle protein kinetics were determined using a model based on stable isotope tracers and arteriovenous blood samples and muscle biopsy.

### (b) Results

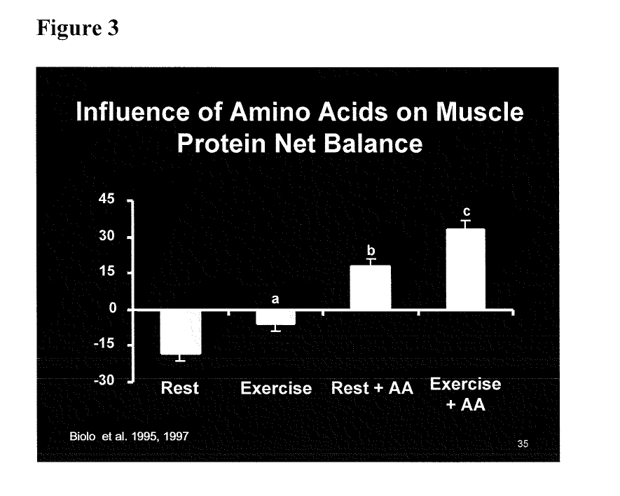

In the basal state net muscle protein balance was negative. Exercise alone improved the net balance as a result of significant stimulation of muscle protein synthesis, but the net balance was still negative. Amino acids alone caused a positive net muscle protein balance as a result of a muscle protein synthesis being stimulated to a greater extent than observed during exercise alone. The combination of amino acids plus exercise further stimulated muscle protein synthesis, such that the net muscle protein balance was increased above the basal value to a greater extent than resulted from either exercise or amino acids alone (FIG. 3).

### (c) Discussion

The results highlight the independent and interactive effects of exercise and amino acids on the rate of muscle protein synthesis. In the basal sate the net loss of muscle protein reflects to fact that the muscle serves as a reservoir of blood amino acids that are in turn used for the synthesis of proteins in other tissues and organs that are essential for life. The stimulatory effect of amino acids is dose dependent, as reflected in Example 4. Whereas exercise alone has a modest stimulatory effect on muscle protein synthesis, it can be viewed as principally “priming” the muscle to respond to the anabolic effects of the amino acids. We conclude from these results that the anabolic effects of amino acids is enhanced by prior exercise.

### Example 3

### Effect of Timing of Amino Acid Intake in Relation to Exercise on Muscle Protein Synthesis

The following study was to determine if ingestion of EAAs before resistance exercise results in a greater anabolic response than supplementation after exercise.

### (a) Methods

Six healthy human volunteers participated in two trials in random order. In both studies a basal period was followed by a strenuous resistance exercise routine. In one case, EAA supplementation was provided immediately before exercise, and in the other case supplementation was provided immediately after exercise. In a separate study, volunteers were provided the supplement 1 hour after completion of the same resistance exercise routine. The exercise bout comprised 10 sets of 8 repetitions of leg press at 80% of 1 repetition maximum (RM), and 8 sets of 8 repetitions of leg extension at 80% of 1 RM. A primed, continuous infusion of 2H5-phenylalanine, femoral arteriovenous catheterization, and muscle biopsies from the vastut lateralis were used to determine the rates of muscle protein synthesis and breakdown, and the net balance between synthesis and breakdown.

### (b) Results

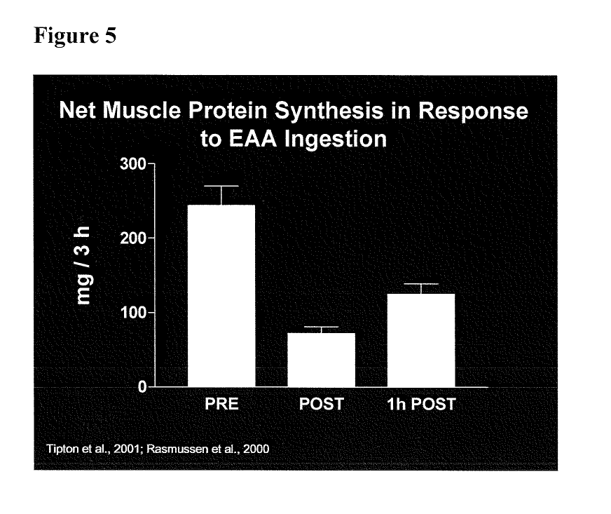

When EAAs were provided before exercise, the stimulation of muscle protein synthesis was significantly (p<0.0002) greater than when provided either immediately after exercise or 1 hour after exercise (FIG. 5). This translated to a greater gain in muscle mass. The effect was attributable at least in part to an increased delivery of amino acids to the exercising muscle.

### (c) Discussion

The timing of ingestion of EAAs in relation to performance of resistance exercise can affect the anabolic response. Therefore, ingestion of an EAA mixture before performance of resistance exercise will be beneficial when incorporated into an overall program of weight reduction and modification of body composition to favor gain in muscle mass and loss of fat mass.

While the compositions and methods discussed herein have been disclosed in connection with certain preferred embodiments, this should not be taken as a limitation to all of the provided details. Modifications and variation of the described embodiments may be made without departing from the spirit and scope of the invention, and other embodiments should be understood to be encompassed in time present disclosures as would be understood by those in ordinary skill in the art.

1. A composition of matter comprising the essential amino acids (EAAs) L-histidine, L-isoleucine, L-leucine, L-lysine, L-methionine, L-phenylalanine, L-valine and L-threonine.

2. The composition of claim 1, wherein the concentration of L-leucine is not less than about 18% of the EAAs and not more than about 40% by weight of the EAAs composition.

3. The composition of claim 2, wherein the concentration of L-histidine ranges from about 8% to about 14% of EAAs by weight of the composition, the concentration of L-isoleucine ranges from about 8% to about 14% of EAAs by weight of the composition, the concentration of L-lysine ranges from about 12% to about 18% of EAAs by weight of the composition, the concentration of L-methionine ranges from about 3% to about 5% of EAAs by weight of the composition, the concentration of L-phenylalanine ranges from about 10% to about 18% of EAAs by weight of the composition, the concentration of L-valine may be between 8-14% of EAAs by weight of the composition, and the concentration of L-threonine ranges from about 12% to about 18% of EAAs by weight of the composition.

4. The composition of claim 1, further comprising an agent selected from the group consisting of: synephrine, Citrus aurantium extract, phenylpropanolamine, caffeine, aspirin, sibutramine, phentermine, diethylpropion, mazindol, phendimetrazine and a combination thereof.

5. The composition of claim 1, wherein the amino acids of the composition are in free form.

6. The composition of claim 1, wherein the amino acids of the composition are in salt form.

7. The composition of claim 1, wherein the composition is incorporated into a food product.

8. A capsule comprising:
a core material comprising L-histidine, L-isoleucine, L-leucine, L-lysine, L-methionine, L-phenylalanine, L-valine and L-threonine; and
a shell wall of material selected from soft gelatin, hard gelatin, and a polymer that encapsulates the core material.

9. The capsule of claim 8, wherein the amino acids of the core material are in free form.

10. The capsule of claim 8, wherein the amino acids of the core material are in salt form.

11. The capsule of claim 8, wherein the core material further comprises at least one excipient.

12. The capsule of claim 8, wherein the core material further comprises an agent selected from the group consisting of: synephrine, Citrus aurantium extract, phenylpropanolamine, caffeine, aspirin, sibutramine, phentermine, diethylpropion, mazindol, phendimetrazine and a combination thereof.

13. A method of use of the composition described above in a program to loose weight and/or to transform body composition to favor increased muscle mass and decreased fat mass.

14. A method to combine ingestion of the composition described above with a resistance exercise routine to result in weight loss and/or transformation of body composition to favor increased muscle mass and strength and decreased fat mass.

15. A method to provide the above composition in 4-5 doses per day in amounts not to exceed 15 g of EAAs per dose to maximize the thermogenic response and the stimulation of muscle protein synthesis over the entire day.

16. A method to provide the composition described above immediately before resistance exercise to maximize the anabolic response to the exercise and to the EAAs.

17. A purified polypeptide, the polypeptide comprising L-histidine residues, L-isoleucine residues, L-leucine residues, L-lysine residues, L-methionine residues, L-phenylalanine residues, L-valine residues, and L-threonine residues.

18. The purified polypeptide of claim 17, wherein the polypeptide is incorporated into a food product.

19. A method for increasing diet induced thermogenesis in a subject, the method comprising administering to the subject a composition comprised of L-histidine, L-isoleucine, L-leucine, L-lysine, L-methionine, L-phenylalanine, L-valine and L-threonine.

20. The method of claim 19, wherein the composition is administered from about 1 hour to about 3 hours after a meal.

21. The method of claim 13, wherein weight loss in the subject is promoted by increased DIT.

22. The method of claim 19, wherein increased DIT leads to decreased body fat and increased lean body mass.
